(19) United States
(12) Patent Application Publication
(10) Pub. No.: US 2005/0276770 A1
(43) Pub. Date: Dec. 15, 2005

(54) Cosmetic lip makeup and/or lipcare composition

(75) Inventors: Vanina Filippi, Paris (FR); Laure Le Chaux, Chevilly-Larue (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 11/139,654

(22) Filed: May 31, 2005

Related U.S. Application Data
(60) Provisional application No. 60/575,826, filed on Jun. 2, 2004.

(30) Foreign Application Priority Data
May 28, 2004 (FR) 04 51055

(51) Int. Cl. A61K 7/025

(52) U.S. Cl. 424/64

(57) ABSTRACT

A cosmetic product may be a lipcare and/or a lip makeup containing, as filler, polyurethane particles and less than 15% by weight of at least one of water and a water-soluble solvent. A cosmetic product may be a lipcare and/or a lip makeup containing, as filler, polyurethane particles in an amount such that a mean gloss of the product is at least 30. A method of preparing a cosmetic product may include employing, as filler, polyurethane particles in the cosmetic product such that a mean gloss of the product is not less than 30. A lip makeup process may include applying such a product to lips.

### CROSS-REFERENCE TO RELATED APPLICATIONS

This non-provisional application claims the benefit of French Application No. 04 51055 filed on May 28, 2004, and U.S. Provisional Application No. 60/575,826 filed on Jun. 2, 2004, the entire disclosures of which are incorporated herein by reference.

### BACKGROUND

The present invention relates to a cosmetic product such as a lip makeup composition and/or a lipcare composition.

In the cosmetics field, development of formulations that not only have good properties in terms of application and comfort, but also satisfactory properties in terms of resistance, especially gloss resistance and migration resistance, is an ongoing objective.

To afford gloss, compounds conventionally used in compositions of this type are viscous oils with a high refractive index, such as, for instance, polybutene or diisostearyl malate, and/or pasty compounds, such as, for instance, polydiglyceryl acyladipate.

Polyurethane particles have been described in general as being useful for preparing cosmetic compositions, especially such compositions having improved sensory properties in terms of softness. Thus, for example, document WO 00/56272 envisions use of polyurethane particles in an aqueous liquid powder composition, which thus has great softness.

### SUMMARY

Under certain conditions, conventional lip makeup and/or lipcare compositions may pose problems regarding tack and/or migration. It is known that migration of compositions may be limited by adding fillers. However, it has been found that adding fillers may generally be detrimental to the gloss of the compositions obtained thereby.

Consequently, there remains at the present time a need for a lip makeup and/or lipcare composition that is acceptable from a sensory point of view and that has satisfactory gloss, while at the same time not substantially migrating.

Exemplary embodiments described herein use a particular type of filler, namely polyurethane particles, that advantageously allows production of compositions that are satisfactory in these terms. Such a result is unexpected.

In exemplary embodiments, use of polyurethane particles as filler in products intended to be applied to the lips may effectively reduce or even prevent migration of the applied composition. In exemplary embodiments, such use may be particularly advantageous especially in terms of gloss and gloss resistance of the product.

Exemplary embodiments of such compositions may also be highly satisfactory in terms of ease of application and/or comfort.

Exemplary embodiments of the present invention may provide a cosmetic product comprising at least one of a lipcare and a lip makeup containing, as filler, polyurethane particles and less than 15% by weight of at least one of water and a water-soluble solvent.

Exemplary embodiments of the present invention may provide a cosmetic product comprising at least one of a lipcare and a lip makeup containing, as filler, polyurethane particles in an amount that is effective to adjust a mean gloss to a value of greater than or equal to 30.

Exemplary embodiments of the present invention may provide a method of preparing a cosmetic product comprising at least one of a lip makeup and a lipcare, a gloss of which is not less than 30, the method comprising: employing, as filler, polyurethane particles in the cosmetic product.

Exemplary embodiments of the present invention may provide a makeup process in which a product as defined above is applied to the lips.

### DETAILED DESCRIPTION OF EMBODIMENTS

For the purposes of the present invention, the term “fillers” is intended to denote particles of any form that are insoluble in a medium of the composition, irrespective of a temperature at which the composition is manufactured, and which remain in the form of particles even after application of the composition. For example, the fillers according to exemplary embodiments of the invention are not film-forming: not capable of forming a continuous film that adheres to a support, for example, especially to lips, for example, not capable of forming a cohesive film, and, better still, not capable of forming a film with cohesion and mechanical properties such that said film may be isolated from said support.

The term “mean gloss” means the gloss as may be conventionally measured using a glossmeter by the following method.

A coat of between 50 μm and 150 μm in thickness of a composition is spread using an automatic spreader onto a Leneta brand contrast card of reference Form 1A Penopac.

The coat of composition covers at least a white background of the card. The deposit is left to dry for 24 hours at a temperature of 30° C., and the gloss at 20° is then measured on the white background using a Byk Gardner brand glossmeter of reference microTri-Gloss.

This measurement (between 0 and 100) is repeated at least three times, and the mean gloss is the mean of the at least three measurements taken.

Advantageously, exemplary embodiments of compositions may have a mean gloss of greater than or equal to 30, 40, 50, 60, 65 or even greater than or equal to 70.

In the present invention, the term “polyurethane particles” is intended to denote particles consisting of a material at least partly of polyurethane type.

In exemplary embodiments, such particles may advantageously be in a crosslinked form.

In exemplary embodiments, the particles may have a mean size ranging from about 4 to about 20 μm, for example, from about 5 to about 15 μm.

In exemplary embodiments, the particles may generally be substantially spherical.

In exemplary embodiments, the polyurethane particles may comprise a copolymer. Said copolymer may comprise trimethylol hexyllactone. For example, the particles may comprise a hexamethylene diisocyanate/trimethylol hexyllactone polymer. Such particles may be commercially available, for example, under the name Plastic Powder D-400® or Plastic Powder D-800® from the company Toshiki.

In exemplary embodiments, the polyurethane particles may be used in an amount such that a mean gloss of the product is greater than or equal to 30, 40, 50, 60, 65 and even greater than or equal to 70.

For example, the particles may range in content from about 0.1% to about 99% by weight relative to a total weight of the product. In exemplary embodiments, the particle content may range from about 0.1% to about 30%, from about 0.1% to about 15% or even from about 0.1% to about 7%.

In exemplary embodiments, the cosmetic product may generally comprise at least one fatty phase. For example, the product may comprise at least one fatty substance that is liquid at room temperature (25° C.) and at atmospheric pressure and/or a fatty substance that is solid at room temperature and atmospheric pressure. The fatty phase may also contain oil structuring agents and gelling agents of organic nature and/or lipophilic organic solvents.

In exemplary embodiments, the fatty phase may comprise, as liquid fatty substance, at least one volatile or non-volatile oil, or a mixture thereof.

For the purposes of the invention, the term “volatile oil” means any oil capable of evaporating on contact with skin or lips in less than one hour, at room temperature and atmospheric pressure. Such volatile oils may comprise volatile cosmetic oils, which are liquid at room temperature, having a non-zero vapor pressure, at room temperature and atmospheric pressure, ranging, for example, from about 0.01 to about 300 mmHg (1.33 Pa to 40 000 Pa), and preferably greater than 0.3 mmHg (30 Pa).

The term “non-volatile oil” means an oil that remains on skin or lips at room temperature and atmospheric pressure for at least several hours and that especially has a vapor pressure of less than 0.01 mmHg (1.33 Pa).

Such volatile or non-volatile oils may be hydrocarbon-based oils, especially of animal or plant origin, and silicone oils, or mixtures thereof. The term “hydrocarbon-based oil” means an oil mainly containing hydrogen and carbon atoms and possibly oxygen, nitrogen, sulfur and/or phosphorus atoms.

As stated previously, such oils may be selected particularly so as to adjust, together with the filler, the degree of gloss desired for the particular cosmetic product.

In exemplary embodiments, the volatile hydrocarbon-based oils may be selected from hydrocarbon-based oils containing from 8 to 16 carbon atoms, and especially branched C₈-C₁₆ alkanes, for instance, C₈-C₁₆ isoalkanes of petroleum origin (also known as isoparaffins), for instance, isododecane (also known as 2,2,4,4,6-pentamethylheptane), isodecane, isohexadecane, and, for example, the oils sold under the trade names Isopar® or Permethyl®, branched C₈-C₁₆ esters, such as isohexyl neopentanoate, and mixtures thereof. Other volatile hydrocarbon-based oils, for instance, petroleum distillates, especially those sold under the name Shell Solt® by the company Shell, may also be used.

In exemplary embodiments, volatile oils that may also be used include volatile silicones, for instance, volatile linear or cyclic silicone oils, especially those with a viscosity ≦8 centistokes (8×10⁻⁶ m²/s) and especially those containing from 2 to 7 silicon atoms. Such silicones may optionally comprise alkyl or alkoxy groups containing from 1 to 10 carbon atoms. Volatile silicone oils that may be used may include octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

In exemplary embodiments, volatile oil content in the product may range from about 0.1% to about 98% by weight relative to the total weight of the product. For example, the volatile oil content may range from about 1% to about 65%, or even from about 2% to about 50% by weight relative to the total weight of the product.

In exemplary embodiments, the non-volatile oils may be selected from non-volatile hydrocarbon-based oils and, where appropriate or desired, non-volatile fluoro oils and/or silicone oils.

In exemplary embodiments, non-volatile hydrocarbon-based oils may include:

hydrocarbon-based oils of animal origin;

hydrocarbon-based oils of plant origin, such as triglycerides consisting of fatty acid esters of glycerol, the fatty acids of which may have varied chain lengths from C₄ to C₂₄, such chains possibly being linear or branched, and saturated or unsaturated. Such oils may be, for example, wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, marrow oil, blackcurrant oil, evening primrose oil, millet oil, barley oil, quinoa oil, rye oil, safflower oil, candlenut oil, passionflower oil or musk rose oil. Further, non-volatile hydrocarbon-based oils may include shea butter or caprylic/capric acid triglycerides, for instance, those sold by the company Stearineries Dubois or those sold under the names Miglyol 810®, 812® and 818® by the company Dynamit Nobel;

synthetic ethers containing from 10 to 40 carbon atoms;

linear or branched hydrocarbons of mineral or synthetic origin, such as petroleum jelly, polydecenes, hydrogenated polyisobutene, such as parleam, and squalane, and mixtures thereof;

synthetic esters, for instance, oils of formula R₁COOR₂ in which R₁ represents a linear or branched fatty acid residue containing from 1 to 40 carbon atoms and R₂ represents a hydrocarbon-based chain, which is especially branched, containing from 1 to 40 carbon atoms, on condition that R₁+R₂≧10, for instance, purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, alcohol or polyalcohol heptanoates, octanoates, decanoates or ricinoleates, for instance, propylene glycol dioctanoate, hydroxylated esters, for instance, isostearyl lactate or diisostearyl malate, polyol esters and pentaerythritol esters;

fatty alcohols that are liquid at room temperature with a branched and/or unsaturated carbon-based chain containing from 12 to 26 carbon atoms, for instance, octyldodecanol, isostearyl alcohol, oleyl alcohol, 2-hexyldecanol, 2-butyloctanol or 2-undecylpentadecanol;

higher fatty acids, such as oleic acid, linoleic acid or linolenic acid, and mixtures thereof.

In exemplary embodiments, non-volatile silicone oils that may be used may be non-volatile polydimethylsiloxanes (PDMS), polydimethylsiloxanes comprising alkyl or alkoxy groups, which are pendant and/or at the end of a silicone chain, such groups each containing from 2 to 24 carbon atoms, phenylsilicones, for instance, phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates, and mixtures thereof.

In exemplary embodiments, a content of the non-volatile oils may range from about 0.01% to about 90% by weight relative to the total weight of the product. For example, the content of the non-volatile oils may range from about 0.1% to about 85%, or even from about 1% to about 70%, by weight relative to the total weight of the product.

In exemplary embodiments, the oils may comprise from about 0.01% to about 99% of the total weight of the product. For example, the oils may comprise from about 0.05% to about 60%, and even from about 1% to about 60%, by weight relative to the total weight of the product.

In the context of the present invention, at least one oil with a molar mass of between about 650 and about 10,000 g/mol, for example, between about 750 and about 7500 g/mol, may advantageously be used. According to exemplary embodiments, the product may comprise an oily phase comprising at least 70% by weight of an oil with a molar mass of between about 650 and about 10,000 g/mol, or even between about 750 and about 7500 g/mol. The oily phase may advantageously comprise more than 80%, and even more than 85%, by weight of an oil with a molar mass of between about 650 and about 10,000 g/mol, or even between about 750 and about 7500 g/mol.

The oil of high molar mass may be selected from:

linear fatty acid esters with a total carbon number ranging from 35 to 70;

hydroxylated esters;

aromatic esters;

esters of fatty alcohols, fatty acids which are branched, or C₂₄-C₂₈;

silicone oils;

oils of plant origin;

and mixtures thereof.

In exemplary embodiments, the oil of high molar mass may be selected, for example, from polybutylenes, hydrogenated polyisobutylenes, polydecenes, hydrogenated polydecenes, vinylpyrrolidone copolyrners, such as PVP/hexadecene copolymer, pentaerythrityl tetrapelargonate, polyglyceryl-2 triisostearate, tridecyl trimellitate, triisoarachidyl citrate, pentaerythrityl tetraisononanoate, glyceryl triisostearate, pentaerythrityl tetraisostearate, glyceryl tris(2-decyl)tetradecanoate, phenyl silicones, sesame oil, and mixtures thereof.

The oils of high molar mass mentioned above are generally glossy oils.

In exemplary embodiments, at least one oil of high molar mass may be used in combination with a polyurethane powder.

Exemplary embodiments of the cosmetic products obtained are particularly satisfactory in terms of gloss.

Advantageously, a ratio between the weight content of the oil of high molar mass and the weight content of polyurethane particles as defined above may be about 1 to 30. For example, the ratio may be about 1 to 20, or even about 1 to 15.

In exemplary embodiments, the cosmetic product may comprise an oily phase with a refractive index of between about 1.46 and about 1.51, which may allow relatively high gloss to be obtained.

In exemplary embodiments, a fatty substance that is liquid at room temperature and at atmospheric pressure may in the product in a proportion from about 0.01% to about 99%, or even from about 0.1% to about 85%, by weight relative to the weight of the fatty phase.

In exemplary embodiments, the product may comprise at least one wax in an amount from about 0.01% to about 70% by weight relative to the total weight of the fatty phase. For example, the wax maybe in an amount from about 0.1% to about 65%, or even from about 1% to about 65%, by weight relative to the total weight of the fatty phase.

For the purposes of the present invention, the term “wax” means a lipophilic fatty compound that is solid at room temperature (25° C.), which undergoes a reversible solid/liquid change of state, which has a melting point of greater than 30° C., which may be up to 200° C., which has a hardness of greater than 0.5 MPa, and which has an anisotropic crystal organization in the solid state. By bringing the wax to its melting point, it is possible to make the wax miscible with oils and to form a microscopically homogeneous mixture, but on returning the temperature of the mixture to room temperature, recrystallization of the wax in the oils of the mixture is obtained.

In exemplary embodiments, waxes that may be used may be compounds that are solid at room temperature and may structure the composition, for example, in the form of a stick. Exemplary waxes may include hydrocarbon-based waxes, fluoro waxes and/or silicone waxes, and may be of plant, mineral, animal and/or synthetic origin. For example, exemplary waxes may have a melting point of greater than about 40° C., or even greater than about 45° C.

In exemplary embodiments, waxes that may be used may be those generally used in cosmetics, such as those of natural origin, for instance, beeswax, camauba wax, candelilla wax, ouricoury wax, Japan wax, cork fiber wax, sugarcane wax, rice wax, montan wax, paraffin, lignite wax or microcrystalline wax, ceresin and ozokerite, and hydrogenated oils, for instance, jojoba oil. The waxes may also comprise synthetic waxes, for instance, polyethylene waxes derived from polymerization or copolymerization of ethylene and Fischer-Tropsch waxes, or alternatively fatty acid esters, for instance, octacosanyl stearate, glycerides that are solid at about 40° C., or even at about 45° C., silicone waxes, for instance, alkyl- or alkoxydimethicones containing an alkyl or alkoxy chain of 10 to 45 carbon atoms, poly(di)methylsiloxane esters that are solid at about 40° C. and with an ester chain containing at least 10 carbon atoms. Exemplary waxes also may include mixtures thereof.

In exemplary embodiments, the amount of wax may be a determining factor for imparting a given level of hardness to the product. Such hardness may be measured by the “cheese-wire” method, comprising cutting a tube of lipstick 12.7 mm in diameter and measuring the hardness at 20° C., using a DFGHS 2® force gauge from the company Indelco-Chatillon, travelling at a speed of 100 mm/minute. The hardness measured is expressed as the shear force (expressed in grams) required to cut a stick under such conditions. According to this method, the hardness of exemplary products formed as a tube of stick type, that is, as a cylinder 12.7 mm in diameter, may range from about 50 to about 300 g. For example, the hardness may range from about 100 to about 250 g, or even from about 100 to about 230 g. When an exemplary product is formulated as a tube of lip pencil type, that is, as a cylinder with a diameter of about 8.3 mm, its hardness may generally be between about 30 and about 250 g. For example, the hardness may be between about 50 and about 240 g, or even between about 50 and about 150 g.

Exemplary embodiments of products in accordance with the invention may generally comprise less than 15% of at least one of water and a water-soluble solvent.

The term “water-soluble solvent” denotes a compound that is liquid at room temperature and miscible with water (water miscibility of greater than 50% by weight at 25° C. and atmospheric pressure).

Exemplary embodiments of water-soluble solvents may also be generally volatile. Such water-soluble solvents may include, for example, lower monoalcohols containing from 1 to 5 carbon atoms, such as ethanol and isopropanol, glycols containing from 2 to 8 carbon atoms, such as ethylene glycol, propylene glycol, 1,3-butylene glycol and dipropylene glycol, C₃ and C₄ ketones and C₂ to C₄ aldehydes.

In exemplary embodiments, the product may have a content of at least one of water and a water-soluble solvent that is less than or equal to 10%, for example, less than or equal to 5%.

In exemplary embodiments, the product is substantially free of water and of water-soluble solvent, i.e. it is an anhydrous product.

In exemplary embodiments, the product may contain another filler besides the polyurethane powder described above. Such fillers may be, for example, talc, zinc stearate, mica, kaolin, polyamide (Nylon®) (Orgasol® from Atochem) powders, polyethylene powders, powders of tetrafluoroethylene (Teflon®) polymers, starch, boron nitride, polymer microspheres, such as those of polyvinylidene chloride/acrylonitrile, for instance, Expancel® (Nobel Industrie), acrylic acid copolymer microspheres (Polytrap® from the company Dow Coming), silicone resin microbeads (for example Tospearls® from Toshiba) and elastomeric organopolysiloxanes.

In exemplary embodiments, cosmetic products may comprise less than 10%, for example, less than 7%, or even less than 5%, by weight of additional filler relative to the total weight of the product.

In exemplary embodiments, the product may comprise at least one coloring agent, for example, that may be in a proportion from about 0.01% to about 40%, for example, from about 0.01% to about 30%, or even from about 0.05% to about 25%, by weight relative to the total weight of the product.

Such coloring agent(s) may be selected from pigments, water-soluble dyes, liposoluble dyes, nacres, flakes, and mixtures thereof.

The term “pigments” should be understood as meaning white or colored, mineral or organic particles, which are insoluble in the liquid hydrophilic phase and which are intended to color and/or opacify the composition. The term “nacres” should be understood as meaning iridescent particles, produced for example by certain molluscs in their shell, or alternatively synthesized.

In exemplary embodiments, the mineral pigments may include titanium dioxide, optionally surface-treated, zirconium oxide or cerium oxide, and also zinc oxide, iron oxide (black, yellow or red) or chromium oxide, manganese violet, ultramarine blue, chromium hydrate and ferric blue, and metal powders, for instance, aluminum powder or copper powder.

In exemplary embodiments, the organic pigments may include carbon black, pigments of D & C type, and lakes based on cochineal carmine or on barium, strontium, calcium or aluminum.

In exemplary embodiments, pigments may be used with an effect, such as particles comprising a natural or synthetic, organic or mineral substrate, for example, glass, acrylic resins, polyester, polyurethane, polyethylene terephthalate, ceramics or aluminas, said substrate being uncoated or coated with metal substances, for instance, aluminum, gold, silver, platinum, copper or bronze, or with metal oxides, for instance, titanium dioxide, iron oxide or chromium oxide, and mixtures thereof.

In exemplary embodiments, nacreous pigments may be selected from mica coated with titanium or with bismuth oxychloride, titanium mica coated with iron oxides, titanium mica coated, for example, with ferric blue or chromium oxide, titanium mica coated with an organic pigment of the above-mentioned type and nacreous pigments based on bismuth oxychloride. In exemplary embodiments, interference pigments, for example, liquid-crystal pigments or multilayer pigments, may also be used.

In exemplary embodiments, water-soluble dyes may include, for example, beetroot juice or methylene blue.

In exemplary embodiments, the product may furthermore comprise any ingredient conventionally used in the fields under consideration and, for example, in cosmetics and dermatology. Such ingredients may be selected, for example, from vitamins, antioxidants, thickeners, trace elements, softeners, sequestering agents, fragrances, acidifying or basifying agents, preserving agents, UV-screening agents and hydrophilic or lipophilic active agents, and mixtures thereof. The amounts of such various ingredients may be as conventionally used in the fields under consideration, for example, from about 0.01% to about 20% of the total weight of the composition.

Needless to say, a person skilled in the art will take care to select such optional additional compound(s), and/or the amount thereof, such that the advantageous properties of exemplary embodiments of the product are not, or are not substantially, adversely affected by the additional compound(s).

In exemplary embodiments, the product may be obtained according to preparation processes conventionally used in cosmetics or dermatology.

In exemplary embodiments, the product may be in the form of a composition that is solid, for example, compacted or cast in a stick or dish form, pasty or liquid.

Advantageously, in solid form, that is, in hard form (not flowing under its own weight), exemplary embodiments may be cast or compacted, for example in stick or dish form.

In exemplary embodiments, the product may be in the form of a lipstick or a lip balm.

In exemplary embodiments, the product may also be in the form of a “liquid gloss.” The term “liquid gloss,” and similarly the terms “liquid lipstick” and “lip gloss,” mean a fluid product intended to be applied to lips and packaged, for example, in a container equipped with an applicator, which may comprise a holding member that also serves as a cap for closing the container, and an application member.

In exemplary embodiments, the product may be in the form of a paste or a cream. In exemplary embodiments, the product may be an oil-in-water or water-in-oil emulsion, an anhydrous, solid or soft gel, or alternatively, may be in the form of loose or compacted powder, and even in a two-phase form. In exemplary embodiments, the product may be in the form of an emulsion.

Exemplary embodiments of products may be in the form of a colored or uncolored product, or in the form of an antisun product. Exemplary embodiments may, for example, contain cosmetic active agents. Exemplary embodiments may then be used as a lipcare or lip-treating base, for instance, lip balms, for protecting lips against cold, sunlight and/or wind. Exemplary embodiments of products may contain, as cosmetic active agents, for example, vitamins A, E, C and B3, provitamins, for instance, D-panthenol, calmative active agents, for instance, α-bisabolol, aloe vera or allantoin, plant extracts or essential oils, protective or restructuring agents, for instance, ceramides, refreshing active agents, for instance, menthol and its derivatives, emollients (cocoa butter), moisturizers (arginine PCA), antiwrinkle active agents and essential fatty acids, and mixtures thereof.

Exemplary embodiments of product may also be in the form of a lip makeup product, for instance, a lipstick or a lip gloss, optionally having care or treating properties.

Needless to say, exemplary embodiments of products should be cosmetically acceptable, that is, nontoxic and able to be applied to human skin and lips.

The examples below are given as illustrations only and are not limiting in any respect.

### EXAMPLE 1

Two lipstick formulations were prepared:
the formulation in accordance with Example 1 incorporates as filler the crosslinked copolymer hexamethylene diisocyanate/trimethylol hexyllactone sold under the name Plastic Powder D4000 by the company Toshiki, and
a control formulation, for comparison, replaces said filler with an equivalent amount of a mixture of fillers conventionally used in lipstick compositions (lauroyllysine/kaolin).

Their compositions were as follows:

| | | Formulation | |
|---|---|---|---|
| | | according to | Control |
| Phase | Starting materials | the invention | formulation |
| A | Diisostearyl malate | 30.99 | 30.99 |
| | Vinylpyrrolidone/hexadecene | 7.74 | 7.74 |
| | copolymer | | |
| | Polyvinyl laurate | 2.62 | 2.62 |
| | Isodecyl neopentanoate | 15.89 | 15.89 |
| | Butylhydroxytoluene | 0.06 | 0.06 |
| A′ | Polybutene | 7.00 | 7.00 |
| | Phenyl trimethicone | 7.60 | 7.60 |
| | (“Belsil PDM 1000” | | |
| | from Wacker) | | |
| | Phenyl trimethicone (“DC | 2.80 | 2.80 |
| | 556” from Dow Corning) | | |
| | Sorbitan oleate | 7.30 | 7.30 |
| B | Polyethylene wax | 6.33 | 6.33 |
| | (“Performalene 500” | | |
| | from New Phase Technologies) | | |
| | Polyethylene wax | 2.10 | 2.10 |
| | (“Performalene 400” | | |
| | from New Phase Technologies) | | |
| | Copolymer | 0.77 | 0.77 |
| | Allyl stearate/vinyl acetate | | |
| | (“Mexomer PQ” from | | |
| | Chimex) | | |
| C | Pigments and nacres | 5.80 | 5.80 |
| D | N-Lauroyl-L-lysine | — | 1.24 |
| | Aluminum silicate | — | 1.76 |
| | Plastic Powder D400 ® | | |
| | | 3.00 | — |
| | Total | 100.00 | 100.00 |

The preparation protocol was as follows:

The pigments and fillers (phase C without the nacres and phase D) were ground using a three-roll mill in the oily phase (phase A+A′). The mixture obtained and the waxes (phase B) were then introduced into a heating pan. The mixture was heated to 98-100° C. while homogenizing using a magnetic bar. The nacres were added five minutes before casting. The paste at 98-100° C. was then poured into a mold at 42° C. Once the paste had set, the mold was placed in a freezer at −18° C. for half an hour. The sticks were then removed from the mold.

These two compositions were tested by three volunteers.

Such tests indicate that the composition according to Example 1 is experienced as being creamier during application than the control formulation. The composition according to Example 1 has better gloss on application, and also keeps better than the control composition. Furthermore, the composition according to the Example 1 migrates less than the control formulation.

### EXAMPLE 2

According to the same protocol as that described in Example 1, the following lipstick composition was prepared:

| | |
|---|---|
| Diisostearyl malate | 29.37 |
| Vinylpyrrolidone/hexadecene copolymer | 7.33 |
| Polyvinyl polylaurate | 2.48 |
| Isodecyl neopentanoate | 15.06 |
| Butylhydroxytoluene | 0.06 |
| Polybutene | 7.00 |
| Phenyl trimethicone (“Belsil PDM 1000” from Wacker) | 7.60 |
| Phenyl trimethicone (“DC 556” from Dow Corning) | 2.80 |
| Sorbitan oleate | 7.30 |
| Polyethylene wax | 6.33 |
| (“Performalene 500” from New Phase Technologies) | |
| Polyethylene wax | 2.10 |
| (“Performalene 400” from New Phase Technologies) | |
| Allyl stearate/vinyl acetate copolymer | 0.77 |
| Pigments and nacres | 6.80 |
| Plastic Powder D400 ® | 5.00 |

On application, the composition of Example 2 was experienced as being both creamier and glossier. Furthermore, the gloss and the comfortable sensation were relatively long-lasting. In addition, the composition of Example 2 had relatively less migration.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention.

1. A cosmetic product comprising at least one of a lipcare and a lip makeup containing, as filler, polyurethane particles and less than 15% by weight of at least one of water and a water-soluble solvent.

2. The product as claimed in claim 1, wherein at least one of a water content and a water-soluble solvent content is not greater than 10% by weight.

3. A cosmetic product comprising at least one of a lipcare and a lip makeup containing, as filler, polyurethane particles in an amount such that a mean gloss of the product is at least 30.

4. The product as claimed in claim 1, wherein a mean gloss of the product is at least 40.

5. The product as claimed in claim 3, wherein the product comprises less than 15% by weight of at least one of water and water-soluble solvent relative to a total weight of said product.

6. The product as claimed in claim 1, wherein said polyurethane particles have a mean size ranging from about 4 to about 20 μm.

7. The product as claimed in claim 1, wherein said polyurethane particles comprise a trimethylol hexyllactone copolymer.

8. The product as claimed in claim 7, wherein said copolymer comprises a hexamethylene diisocyanate/trimethylol hexyllactone copolymer.

9. The product as claimed in claim 1, wherein said polyurethane particles range from about 0.1% to about 99% by weight relative to a total weight of said product.

10. The product as claimed in claim 1, wherein the product comprises at least one fatty phase.

11. The product as claimed in claim 10, wherein the at least one fatty phase comprises at least one oil selected from polybutenes, hydrogenated polyisobutylenes, polydecenes, hydrogenated polydecenes, vinylpyrrolidone copolymers, polyvinylpyrrolidone/hexadecene copolymer, pentaerythrityl tetrapelargonate, polyglyceryl-2 triisostearate, tridecyl trimellitate, triisoarachidyl citrate, pentaerythrityl tetraisononanoate, glyceryl triisostearate, pentaerythrityl tetraisostearate, glyceryl tris(2-decyl)tetradecanoate, phenyl silicones, plant oils, and sesame oil.

12. The product as claimed in claim 1, wherein the product comprises at least one coloring agent in a proportion ranging from about 0.01% to about 40% by weight relative to a total weight of said product.

13. The product as claimed in claim 12, wherein said coloring agent comprises at least one selected from pigments, nacres, water-soluble dyes and liposoluble dyes, and mixtures thereof.

14. The product as claimed in claim 1, wherein the product is in the form of a product cast in at least one of a stick form and a dish form.

15. The product as claimed in claim 1, wherein the product is in the form of a liquid gloss.

16. A method of preparing a cosmetic product comprising at least one of a lip makeup and a lipcare, a gloss of which is not less than 30, the method comprising:
employing, as filler, polyurethane particles in the cosmetic product.

17. The method as claimed in claim 16, wherein the polyurethane particles are in an amount such that a mean gloss of the product is at least 30 and the cosmetic product comprises less than 15% by weight of at least one of water and water-soluble solvent relative to a total weight of said cosmetic product.

18. A lip makeup process comprising applying the product of claim 1 to lips.

19. The product as claimed in claim 2, wherein at least one of a water content and a water-soluble solvent content is not greater than 5% by weight.

20. The product as claimed in claim 4, wherein the mean gloss of the product is at least 50.

21. The product as claimed in claim 20, wherein the mean gloss of the product is at least 60.

22. The product as claimed in claim 23, wherein the mean gloss of the product is at least 70.

23. The product as claimed in claim 5, wherein the product comprises less than 10% by weight of at least one of water and water-soluble solvent relative to the total weight of said product.

24. The product as claimed in claim 23, wherein the product comprises less than 5% by weight of at least one of water and water-soluble solvent relative to the total weight of said product.

25. The product as claimed in claim 6, wherein said polyurethane particles have a mean size ranging from about 5 to about 15 μm.

26. The product as claimed in claim 9, wherein said polyurethane particles range from about 0.1% to about 30% by weight relative to the total weight of said product.

27. The product as claimed in claim 26, wherein said polyurethane particles range from about 0.1% to about 15% by weight relative to the total weight of said product.

28. The product as claimed in claim 27, wherein said polyurethane particles range from about 0.1% to about 7% by weight relative to the total weight of said product.

29. The product as claimed in claim 3, wherein a mean gloss of the product is at least 40.

30. The product as claimed in claim 3, wherein said polyurethane particles have a mean size ranging from about 4 to about 20 μm.

31. The product as claimed in claim 3, wherein said polyurethane particles comprise a trimethylol hexyllactone copolymer.

32. The product as claimed in claim 3, wherein said copolymer comprises a hexamethylene diisocyanate/trimethylol hexyllactone copolymer.

33. The product as claimed in claim 3, wherein said polyurethane particles range from about 0.1% to about 99% by weight relative to a total weight of said product.

34. The product as claimed in claim 29, wherein a mean gloss of the product is at least 50.

35. The product as claimed in claim 34, wherein a mean gloss of the product is at least 60.

36. The product as claimed in claim 35, wherein a mean gloss of the product is at least 70.

37. The product as claimed in claim 30, wherein said polyurethane particles have a mean size ranging from about 5 to about 15 μm.

38. The product as claimed in claim 33, wherein said polyurethane particles range from about 0.1% to about 30% by weight relative to a total weight of said product.

39. The product as claimed in claim 38, wherein said polyurethane particles range from about 0.1% to about 15% by weight relative to a total weight of said product.

40. The product as claimed in claim 39, wherein said polyurethane particles range from about 0.1% to about 7% by weight relative to a total weight of said product.
